(19) United States
(12) Patent Application Publication
(10) Pub. No.: US 2011/0183329 A1
(43) Pub. Date: Jul. 28, 2011

(54) PROTEIN PHOSPHATASE-1 INHIBITOR-1 POLYMORPHISM AND METHODS OF USE

(75) Inventors: Anand Pathak, Ann Arbor, MI (US); Evangelia Kranias, Cincinnati, OH (US); Guoli Chen, Mason, OH (US)

(73) Assignee: THE UNIVERSITY OF CINCINNATI, Cincinnati, OH (US)

(21) Appl. No.: 12/911,634

(22) Filed: Oct. 25, 2010

Related U.S. Application Data
(63) Continuation of application No. 12/810,575, now abandoned.
(60) Provisional application No. 61/010,784, filed on Jan. 11, 2008.

(51) Int. Cl. C12Q 1/68; C12N 5/10; C12N 15/85; C07K 14/47; C07H 21/04

(52) U.S. Cl. 435/6.11; 435/6.1; 435/6.12; 435/325; 435/320.1; 530/350; 536/23.5

(57) ABSTRACT

A method for diagnosing the presence of a G147D polymorphism of human phosphatase-1 inhibitor-1 protein is provided, wherein the method involves determining the presence of the polymorphism in a biological sample from a human subject by means which detect the presence of the polymorphism, wherein the presence of the polymorphism is indicative of a predisposition to impaired functioning of the β-adrenergic signaling pathway.

### CROSS REFERENCE TO RELATED APPLICATION

This application is a continuation of U.S. application Ser. No. 12/810,575, which is the U.S. National Phase of PCT international Application Ser. No. PCT/US2009/000208, filed Jan. 12, 2009 and published in English on Aug. 6, 2009 as publication WO 2009/097080 A1, which claims the benefit of U.S. Provisional Application Ser. No. 61/010,784, filed Jan. 11, 2008. The entire contents of the aforementioned patent applications are hereby incorporated herein by this reference.

Any and all references cited in the text of this patent application, including any U.S. or foreign patents or published patent applications, International patent applications, as well as, any non-patent literature references, including any manufacturer's instructions, are hereby expressly incorporated herein by reference.

### BACKGROUND OF THE INVENTION

As the leading cause of morbidity and mortality worldwide, heart failure has been intensively studied in an effort to elucidate underlying mechanisms and identify new potential therapeutic targets. The molecular mechanisms underlying the pathogenesis of heart failure involve diverse and multiple interacting pathways. Among them, the β-adrenergic receptor signaling axis is one of the most prominent and well-characterized pathways in regulation of cardiac function, and disturbances of this cascade contribute to development and progression of heart failure.

The functional effects of β-adrenergic agonists on cardiomyocyte contractility are mediated by key intracellular target phosphoproteins. In particular, agonist binding to β-adrenergic receptors increases adenylyl cyclase activity, which activates protein kinase A (PKA), through elevated levels of cAMP. PKA-mediated phosphorylation of sarcoplasmic reticulum (SR) proteins, such as ryanodine receptor (RyR) and phospholamban (PLN), troponin I and C-protein, in the myofilaments, and plasma membrane L-type calcium channel increases contractility through multiple mechanisms, including via SERCA2a, leading to enhanced calcium cycling and myofilament sensitivity. Opposing the PKA axis are protein phosphatases that dephosphorylate these regulatory proteins and play an essential role in maintaining biochemical and functional phosphorylation homeostasis. Thus, a dynamic balance between phosphorylation by protein kinases and dephosphorylation by protein phosphatases is necessary for normal cardiac pumping function. However, in experimental and human heart failure, the adrenergic pathway is depressed, while phosphatase activity is elevated, tilting this fine balance in favor of dephosphorylation of key phosphoproteins. As a major protein phosphatase in the heart, protein phosphatase-1 (PP1) has emerged as an important player in cardiac function under physiological and pathological conditions, due to its involvement in regulation of the phosphorylation status of important cardiac proteins, such as, for example, PLN and RyR. Thus, regulation of its activity has been suggested as a potential target for the rescue of failing heart function.

An additional layer of complexity to PKA-PP1 is added by regulation of this phosphatase activity by endogenous proteins. One such factor, inhibitor-1, is itself phosphorylated by PKA or β-adrenergic stimulation on threonine 35 and potentially inhibits protein phosphatase-1 activity, allowing PKA phosphorylation to propagate and increase cardiac contractility in an unopposed manner. Indeed, expression of a truncated (amino acids 1-65) and constitutively phosphorylated form (T35D) of inhibitor-1 enhances cardiac contractility by favoring specific phosphorylation of PLN. More meaningfully, this model exhibited a higher resistance to cardiac remodeling and heart failure induced by chronic over pressure-loading than wild-type controls. Further, gene transfer of the active inhibitor-1 form in pre-existing heart failure restored function and attenuated the progression of cardiac remodeling. In contrast, decreased activity and/or reduced protein level of inhibitor-1 are associated with cardiac dysfunction in animal models and in human heart failure, suggesting that defects in inhibitor-1 activity may contribute to depressed cardiac function.

Inhibitor-1 is a protein comprising 171 amino acids. To date, efforts have been primarily focused on clarifying the significance of its N-terminal region. As described above, the threonine 35 (T35) can be phosphorylated by PKA to prompt its inhibitory effect on protein phosphatase-1. In addition, inhibitor-1 is phosphorylated at its serine 67 (S67) and threonine 75 (T75) residues by PKC, which blunts its inhibition of protein phosphatase-1. Further, phosphorylation of either of these two PKC sites may attenuate the stimulatory effects of the PKA pathway in cardiac myocytes.

By sequencing the inhibitor-1 gene in 352 normal subjects and 959 heart failure patients, the present inventors have identified a novel C-terminal polymorphism of human inhibitor-1, entailing substitution of aspartic acid for glycine at amino acid 147 (G147D). Further, overexpression of G147D inhibitor-1 in vitro blunted the response of adult rat cardiomyocytes to β-adrenergic stimulation by interfering with phosphorylation of PLN. Thus, the presence of the G147D polymorphism of inhibitor-1 in a human subject is associated with impaired functioning of the β-adrenergic signaling pathway and may contribute to cardiac dysfunction in human heart failure patients.

Moreover, detection of the presence of the G147D polymorphism is a useful indicator of an individual who would benefit from targeted gene therapy. Given that decreasing phosphatase activity in the sarcoplasmic reticulum calcium transport pathway (“SR Ca-transport pathway”) favorably impacts β-adrenergic responsiveness, the proteins of this pathway (including, but not limited to, RyR, PLN, troponin I, C-protein, plasma membrane L-type Ca channels, and inhibitor-1) provide suitable targets for therapeutic treatment of subjects suffering from cardiac disorders, including heart failure. Gene therapy having a therapeutic effect on proteins of the SR Ca-transport pathway provides a method for administering into heart cells, or cardiomyocytes, an agent that favorably modulates phosphatase activity, including type 1 phosphatase activity, in the cells.

### SUMMARY OF THE INVENTION

Accordingly, the present invention is directed to the detection and use of polymorphisms of human protein phosphatase-1 inhibitor-1. In a particular embodiment, the invention provides for the detection and use of a G147D polymorphism of human protein phosphatase-1 inhibitor-1.

In one aspect, the invention provides a method for diagnosing the presence of a polymorphism of human phosphatase-1 inhibitor-1 protein which causes a predisposition to impaired functioning of the β-adrenergic signaling pathway, wherein the method comprises determining the presence of the polymorphism in a biological sample from a human subject by means which detect the presence of the polymorphism, wherein the polymorphism comprises aspartic acid at position 147 of the phosphatase-1 inhibitor-1 protein and wherein the presence of the polymorphism is indicative of said predisposition.

In one embodiment of the invention, the polymorphism can result in a phosphatase-1 inhibitor-1 having the amino acid sequence of SEQ ID NO:14, i.e., wherein the glycine (G) at residue 147 in the wildtype protein is substituted by aspartic acid (D) in the polymorphism form.

In still another embodiment, the detecting means comprises analyzing DNA, RNA, or protein from the biological sample.

In yet another embodiment, the detecting means can include carrying out a polymerase chain reaction in a mixture containing the biological sample and a pair of primers, wherein the primers are selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12 or complements thereof, under conditions suitable for amplifying a polynucleotide.

In still another embodiment, the detecting means further includes the step of sequencing the polynucleotide to detect the presence or absence of the polymorphism.

In another aspect, the present invention provides an isolated phosphatase-1 inhibitor-1 protein having the amino acid sequence of SEQ ID NO:14, i.e., a phosphatase-1 inhibitor-1 protein wherein the glycine (G) normally at residue 147 is substituted by aspartic acid (D) at the same position.

In still another aspect, the invention provides an isolated DNA comprising a nucleic acid sequence encoding a phosphatase-1 inhibitor-1 having the amino acid sequence of SEQ ID NO:14, as well as cells transfected with the isolated DNA and to vector comprising the isolated DNA.

In yet another aspect, the present invention provides a method for detecting a polymorphism of human protein phosphatase-1 inhibitor-1, wherein the polymorphism is associated with impaired functioning of the β-adrenergic signaling pathway in a human subject, wherein the method comprises analyzing a protein phosphatase-1 inhibitor-1 gene or gene expression product from a biological sample of the human subject.

In one embodiment of the invention, the method for detecting a human protein phosphatase-1 inhibitor-1 polymorphism can involve comparing the sequence of one or more wild-type protein phosphatase-1 inhibitor-1 gene sequences with the sequence of the protein phosphatase-1 inhibitor-1 gene obtained in a biological sample, including, for example, the protein phosphatase-1 inhibitor-1 gene encoding the amino acid sequence of SEQ ID NO:14

In a still further aspect, the present invention provides a method for determining whether a human subject who is a candidate for a therapy that mitigates an impairment of g-adrenergic responsiveness caused by a defective phosphatase-1 inhibitor-1 gene, the method comprising the steps of:

(1) obtaining a biological sample from the human subject, wherein the biological sample comprises a nucleic acid molecule encoding a phosphatase-1 inhibitor-1 gene; and

(2) detecting the presence or absence of a genetic polymorphism of the phosphatase-1 inhibitor-1 gene of said human subject,

wherein the presence of the polymorphism identifies a human subject who is a candidate for a therapy that mitigates an impairment of β-adrenergic responsiveness caused by a defective phosphatase-1 inhibitor-1 gene.

In one embodiment, the genetic polymorphism of the phosphatase-1 inhibitor-1 gene comprises aspartic acid (D) in place of glycine (G) at position 147 of the phosphatase-1 inhibitor-1 protein.

In another embodiment, the therapy is to administer gene therapy such that the impairment of the β-adrenergic responsiveness is mitigated. In a further aspect, the gene therapy can include gene replacement therapy of the defective phosphatase-1 inhibitor-1 gene with a non-defective phosphatase-1 inhibitor-1 gene, thereby mitigating the impairment of the β-adrenergic responsiveness caused by the defective phosphatase-1 inhibitor-1 gene. In another aspect, the gene therapy can include administering a therapeutically effective amount of a vector encoding SERCA2a, thereby mitigating the impairment of β-adrenergic responsiveness caused by the defective phosphatase-1 inhibitor-1 gene.

In yet another embodiment, the therapy can include the administering of a therapeutically effective amount of an agent to mitigate the impairment of β-adrenergic responsiveness. In certain aspects, the agent can be an inhibitor of the activity or expression of protein phosphatase-1, thereby mitigating the impairment of β-adrenergic responsiveness caused by the defective phosphatase-1 inhibitor-1 gene. In certain additional aspects, the agent can be an inhibitor of the activity or expression of phospholamban, thereby mitigating the impairment of β-adrenergic responsiveness caused by the defective phosphatase-1 inhibitor-1 gene.

In certain embodiments, the inhibitor agent can be a small molecule, an antibody, a siRNA, a miRNA, or an antisense RNA.

In another aspect, the invention provides a kit for detecting the presence or absence of a polymorphism of phosphatase-1 inhibitor-1, the kit comprising a pair of primers, each primer having at least 95% identity to a primer selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12 or complements thereof. In one embodiment, the primers are selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified.

### BRIEF DESCRIPTION OF THE DRAWINGS

The following Detailed Description, given by way of example, but not intended to limit the invention to specific embodiments aspects described, may be understood in conjunction with the accompanying drawings, which are incorporated herein by reference. Various features and aspects of the present invention will now be described by way of non-limiting examples and with reference to the accompanying drawings, in which:

FIG. 1. A single nucleotide transition from G to A at position 440, which resulted in the transition of glycine (G) to aspartic acid (D) at position 147 in exon 6 of inhibitor-1 was identified.

FIG. 2. Isolated adult rat cardiomyocytes were infected with adenoviruses expressing GFP as control (empty), G147 inhibitor-1 or D147 inhibitor-1. (A) Images of infected cardiomyocytes and GFP expression. (B) Representative western blots display expression levels of inhibitor-1, SERCA 2a, PLN, CSQ, and L-type Ca-channel (LTCC).

FIG. 3. Contractile parameters were measured in infected adult rat cardiomyocytes. (A) Representative traces for contractility. (B) Fractional shortening (FS %). (C) Contracting velocity (−dL/dT). (D) Relaxing velocity (+dL/dT). *:p<0.05, G147D vs. GFP pr WT; #:p<0.05, in −Isoproterenol vs. +Isoproterenol.

FIG. 4. Ca kinetics were measured in infected adult rat cardiomyocytes. (A) Ca transient amplitude. (B) Tau for Ca transient decay. *:p<0.05, G147D vs. GFP pr WT; #:p<0.05, in −Isoproterenol vs. +Isoproterenol.

FIG. 5. Phosphorylation levels of PLN on serine 16 and threonine 17 were examined in the 3 groups of infected adult rat cardiomyocytes in the presence of isoproterenol. (A) Representative western blots. (B) Ratio of phosphorylated S16 PLN (S16P-PLN)/total PLN. (C) Ratio of phosphorylated T17 PLN (T17P-PLN)/total PLN. (D) Ratio of total PLN/CSQ. *:p<0.05, G147D vs. GFP or WT.

FIG. 6. Phosphorylation levels of troponin I (TnI) and ryanodine receptor (RyR) were examined in infected adult rat cardiomyocytes in the presence of isoproterenol. Representative western blots are shown for phosphorylated and total troponin I (A) and RyR (C). The ratios of phosphorylated/total Tn1 (B) and phosphorylated/total RyR (D) were calculated. *:p<0.05, G147D vs. GFP or WT.

### DETAILED DESCRIPTION OF THE INVENTION

The following is a list of definitions for terms used herein.

The term “polymorphism” is used herein to refer to genetic variations of a nucleic acid sequence occurring in a statistically significant percentage of a population of subjects. The G147D polymorphism of the present invention is a mutation which results in the replacement of glycine with aspartic acid at position 147 of the amino acid sequence of phosphatase-1 inhibitor-1 protein. The G147D polymorphism is illustrated in the nucleotide sequence of SEQ ID NO: 16 and the polypeptide sequence of SEQ ID NO:14 herein.

The terms “phosphatase-1 inhibitor-1 protein” and “inhibitor-1 protein,” as used herein, refer to a protein described, for example, by GenBank Accession No. NM_{—}006741 (accessed Jan. 6, 2008), which regulates cardiac contractility by inhibiting the activity of protein phosphatase-1.

In the context of the phosphatase-1 inhibitor-1 protein, the term “wild type” and its abbreviation “WT” refer to the nucleotide sequence of SEQ ID NO:15 encoding phosphatase inhibitor-1 protein, subunit 1A, and the polypeptide of SEQ ID NO:13 and any other nucleotide sequence that encodes an inhibitor-1 protein having the same functional properties and binding affinities as the aforementioned polypeptide sequences, such as allelic variants.

The term “β-adrenergic signaling pathway,” as used herein, refers to the route or pathway by which beta-adrenergic agonists, such as isoproterenol, stimulate contractility. This includes the binding of the beta agonist to its receptor on the outer cell membrane, increases in cyclic AMP levels, activation of PKA and phosphorylation of its substrates that increase cardiac function. The β-adrenergic signaling pathway is well-characterized in the art. See, for example, Chakraborti S, Calcium signaling phenomena in heart diseases: a perspective, Mol Cell Biochem., 2007 April; 298(1-2):1-40, which is incorporated herein by reference.

The term “β-adrenergic responsiveness,” as used herein, refers to the regulation of cardiac contractility and those related physiological responses involved in cardiac function (e.g., calcium cycling via the sarcoplasmic reticulum) which are triggered by the binding of an appropriate agonist to an adrenergic receptor. The term “β-adrenergic responsiveness” is meant to encompass the physiological processes of the β-adrenergic signaling pathway, but also other functions/processes involved in controlling the action resulting from the binding of an agonist to its cognate β-adrenergic receptor. β-adrenergic responsiveness specifically encompasses SERCA2a (sarcoplasmic reticulum Ca²⁺-ATPase activity) and its related processes and functions.

The term “mitigate,” as use herein as in “identifying a human subject who is a candidate for a therapy that mitigates an impairment of β-adrenergic responsiveness caused by a defective phosphatase-1 inhibitor-1 gene,” the term means to alleviate or even fully reduce a condition, disease, or symptom thereof, such as the condition of having an impairment in β-adrenergic responsiveness.

“Biological sample” refers to any sample obtained from a subject that contains genetic material that can be extracted to provide genomic information relating to the subject (including, but not limited to, DNA, RNA, proteins, antibodies, and the like). The sample may, for example, comprise blood, buccal swab, hair, paraffin-embedded tissue, biopsy tissue, or any other biological sample from which genetic material may be extracted. In one embodiment of the invention, the sample comprises genomic DNA capable of extraction and purification.

The term “subject,” as used herein, means any mammalian subject, including humans.

The terms “polymerase chain reaction” and “PCR,” as used herein, refer to a molecular biology technique for amplifying a DNA (deoxyribonucleic acid) fragment in vitro via enzymatic replication.

The term “primer,” as used herein, refers to short oligonucleotides capable of hybridizing to target DNA. In PCR, primers are used to direct replication of the DNA fragment of choice.

In describing the sequences identified herein, reference is made to the sense strand of the sequence for convenience. However, as recognized by the skilled artisan, nucleic acid molecules may be complementary double stranded molecules and thus reference to a particular site on the sense strand refers as well to the corresponding site on the complementary antisense strand. Thus, with respect to the primers disclosed herein, one skilled in the art will appreciate that the sense strand sequences are given in SEQ ID NOS:1-12, and that complements of those sequences would work equally well.

The term “polynucleotide” refers to an organic polymer molecule comprised of nucleotide monomers.

The term “transfected,” refers to insertion of a DNA or vector into a target cell. Methods of transfecting cells are well known to the skilled artisan.

“Isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide.

The term “vector,” as used herein, refers to a DNA molecule used to transfer foreign genetic material into another cell. Various types of vectors are known in the art, including plasmids, cosmids, bacteriophages, viruses, and artificial chromosomes and the like. A number of virus and virus-like particles are suitable for use as vectors in the present invention, including but not limited to adeno-associated virus, adenovirus, and lentivirus.

The term “gene expression product,” as used herein, refers to the biochemical material, including RNA or protein, resulting from the expression of a gene.

The term “detection assay,” as used herein, refers to any assay configured to specifically identify the presence or absence of the polymorphism in human protein phosphatase-1 inhibitor-1. Examples of suitable detection methods are illustrated below and include, but are not limited to allele-specific antibody-based assays, ELISA, PCR based methods, nucleic acid hybridization, nucleic acid sequencing, DNA microarrays, DNA bead arrays, mass spectrometry, primer extension, branched DNA, pyrosequencing methods and enzymatic cleavage methods.

The term “therapy,” as used herein, refers to any suitable therapeutic means which may be administered to mitigate or fully repair or restore an impairment or disease condition or symptom thereof. The particular form of therapy is not limited to any particular type and can include, for example, gene therapy or intervention with an agent, either of which may be suitable to mitigate or restore an impairment of interest, e.g., an impairment of the β-adrenergic signaling pathway caused by a defective phosphatase-1 inhibitor-1 gene, e.g., the G147D mutation or polymorphism. The target of the gene therapy or other therapy can be any suitable target, such as the defective phosphatase-1 inhibitor-1 gene itself, or enhancing/inhibiting the expression and/or activity of another target, e.g., protein phosphatase I, phospholamban or SERCA2a, which indirectly results in the mitigation or restoration of the impairment, e.g., the impairment of β-adrenergic responsiveness. These targets can include, but are not limited to, RyR, PLN, troponin I, C-protein, plasma membrane L-type Ca channels, SERCA2 and inhibitor-1.

In one embodiment, gene therapy can be used to replace the defective inhibitor-1 gene by administering into the heart cells of a subject a therapeutically effective amount of a nucleic acid encoding a wild-type phosphatase-1 inhibitor-1 protein. In another embodiment, gene therapy can be used to introduce additional copies of SERCA2a to mitigate the negative effect on calcium cycling and cardiac contractility caused by a defective phosphatase-1 inhibitor-1 mutant (e.g., the G147D polymorphism). In this embodiment, a therapeutically effective amount of a vector encoding SERCA2a can be administered into heart cells of a subject, thereby restoring the impaired β-adrenergic responsiveness due to a defective phosphatase-1 inhibitor-1, e.g., a G147D polymorphism). Methods for administration of inhibitor-1 gene replacement therapy are disclosed, for example, in WO publications WO/2006/029319, published Mar. 16, 2006 and WO/2007/100465, published Sep. 7, 2007, which documents are incorporated by reference in their entirety. Methods for administering SERCA2a by gene therapy are known and are disclosed, for example, in US Published Application No. 2005/0095227, which is incorporated herein by reference.

The term “heart disorder” refers to a structural or functional abnormality of the heart that impairs its normal functioning. For example, the heart disorder can be heart failure, ischemia, myocardial infarction, congestive heart failure, arrhythmia, transplant rejection and the like. The term includes disorders characterized by abnormalities of contraction, abnormalities in Ca²⁺cycling, and disorders characterized by arrhythmia.

The term “heart failure” refers to any of a number of disorders in which the heart has a defect in its ability to pump adequately to meet the body's needs. In many cases, heart failure is the result of one or more abnormalities at the cellular level in the various steps of excitation-contraction coupling of the cardiac cells. One such abnormality is a defect in SR function. As used herein, the term “heart cell” refers to a cell which can be: (a) part of a heart present in a subject, (b) part of a heart which is maintained in vitro, (c) part of a heart tissue, or (d) a cell which is isolated from the heart of a subject. For example, the cell can be a cardiomyocyte.

The term “obtaining,” as a primer of the invention, is intended to include purchasing, synthesizing or otherwise acquiring the elements of the invention.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### Study Subjects

The human study protocols were approved by the institutional review board of the University of Cincinnati, and subjects provided written informed consent. Subjects with heart failure were aged 18-80 yr and had New York Heart Association class II-IV heart failure (see Table 1).

TABLE 1
Clinical Characteristics in Heart Failure Patients by Race

| | Whites | | Blacks | | |
|---|---|---|---|---|---|
| Variable | N | Mean ± SD | N | Mean ± SD | P Value |
| Age at onset of heart failure (yrs) | 671 | 55.73 ± 12.78 | 288 | 52.66 ± 13.33 | 0.001 |
| Duration of heart failure (yrs) | 506 | 11.56 ± 6.28 | 198 | 9.59 ± 5.91 | <0.001 |
| Weight (kg) | 656 | 85.85 ± 21.46 | 281 | 90.68 ± 25.99 | 0.006 |
| Height (cm) | 649 | 172.56 ± 14.68 | 272 | 171.58 ± 10.42 | 0.314 |
| Systolic BP (mmHg) | 540 | 117.73 ± 21.25 | 231 | 119.88 ± 23.65 | 0.234 |
| Diastolic BP (mmHg) | 540 | 72.96 ± 13.49 | 230 | 75.00 ± 14.55 | 0.061 |
| LVEDD (cm) | 526 | 6.43 ± 1.18 | 254 | 6.12 ± 1.02 | <0.001 |
| LVESD (cm) | 502 | 5.08 ± 1.38 | 251 | 4.80 ± 1.29 | 0.007 |
| Septal Wall (cm) | 476 | 1.07 ± 0.28 | 246 | 1.13 ± 0.28 | 0.018 |
| Posterior Wall (cm) | 476 | 1.01 ± 2.44 | 246 | 1.04 ± 2.66 | 0.239 |
| LVEF (%) | 422 | 27.62 ± 1.07 | 150 | 33.09 ± 14.96 | <0.001 |
| Fractional Shortening (%) | 502 | 21.74 ± 11.19 | 251 | 22.54 ± 11.81 | 0.368 |
| MVO2 | 409 | 17.39 ± 5.83 | 137 | 15.74 ± 5.08 | 0.003 |
| Predicted MVO2 | 389 | 67.97 ± 24.83 | 132 | 68.13 ± 23.58 | 0.949 |

| Variable | N | % | N | % | P Value |
|---|---|---|---|---|---|
| Male | 464 | 69.2 | 157 | 54.5 | <0.001 |
| Primary Diagnosis | | | | | |
| Idiopathic Cardiomyopathy | 332 | 52.2 | 175 | 63.4 | |
| Coronary Atery Disease | 222 | 34.9 | 44 | 15.9 | |
| Other | 82 | 12.9 | 57 | 20.7 | <0.001 |
| Cardiac related death or received transplant | 213 | 31.8 | 54 | 18.8 | <0.001 |
| Other risk factors or coexisting conditions | | | | | |
| Hypertension | 313 | 47.2 | 227 | 79.4 | <0.001 |
| Diabetes mellitus | 199 | 33.5 | 93 | 34.8 | 0.703 |
| Hypercholesterolemia | 166 | 31.4 | 56 | 25.3 | 0.099 |
| Tobacco use | | | | | |
| Present user | 294 | 63.4 | 144 | 68.9 | |
| Past only | 96 | 20.7 | 36 | 17.2 | |
| Never | 74 | 15.9 | 29 | 13.9 | 0.374 |
| Medications at entry | | | | | |
| Beta-blocker | 453 | 70.6 | 228 | 81.7 | <0.001 |
| ACE inhibitor | 487 | 88.9 | 223 | 93.7 | 0.035 |
| ARB | 80 | 38.8 | 40 | 34.8 | 0.472 |
| Adlo blocker | 83 | 32.8 | 39 | 31.5 | 0.792 |

### Genotyping

Exons 1-6 of inhibitor-1 (according to National Center for Biotechnology Information, PPP1R1A protein phosphatase 1, regulatory (inhibitor) subunit 1A [Homo sapiens], Gene ID: 5502, in chromosome 12) were screened. Primer sequences, used for amplification and sequencing, included the following: for exon 1, 5′-CAAAACTCCGAGGACACTGAGGTATC (SEQ ID NO:1)(upstream fragment) and 5′-CAAAAGGGCGCACTGCTAAGGGAG (SEQ ID NO:2) (downstream fragment); for exon 2, 5′-GGCCTCCAGCTGCATTAACAT (SEQ ID NO:3) (upstream fragment) and 5′-GTTCTTCTTCCATCTAGCGCC (SEQ ID NO:4) (downstream fragment); for exon 3,5′-CAAGGACGGGACTAGATGCAGAG (SEQ ID NO:5) (upstream fragment) and 5′-GTTCACTCATGCACATTTGGG (SEQ ID NO:6) (downstream fragment); for exon 4, 5′-TTGCTCACCATTGTAGTCTCC (SEQ ID NO:7) (upstream fragment) and 5′-CCCAGCTAGTCAGTGGCAAAA (SEQ ID NO:8) (downstream fragment); for exon 5, 5′-CCTCCTTGTTCAGATCTCAGTG (SEQ ID NO:9) (upstream fragment) and 5′-CCTTGAGACAGTTTTTGCC (SEQ ID NO:10) (downstream fragment); and for exon 6,5′-CCCTGTTGGTCTTGCCTGATTG (SEQ ID NO:11) (upstream fragment) and CCTACTGCTCTCACCCATTCCA (SEQ ID NO:12) (downstream fragment). For polymorphism discovery and genotyping bidirectional automated sequencing, an Applied Biosystems ABI 3130-x1 Genetic Analyzer in 96-well formats was used.

Sequences were confirmed in 98% of samples and were aligned with a reference sequence using Seqscape v2.5.

### Identification of a Genetic Variant in the Human Inhibitor-1 Gene

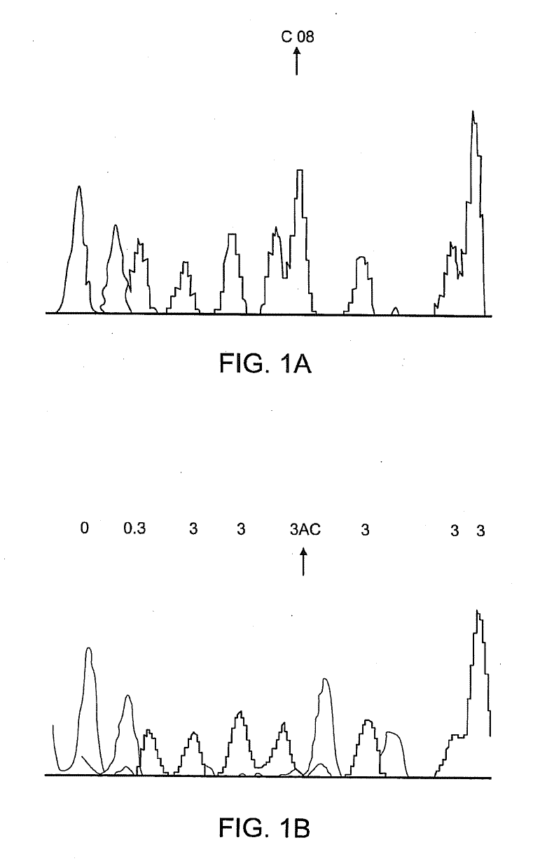

The protein phosphatase-1 inihibitor-1 gene was sequenced in 963 unrelated patients with heart failure recruited from the University Hospital and Cincinnati Heart Failure/Transplant Program, and a single nucleotide transition from G>A was identified at position 440 (c.440G>A), which resulted in the transition of glycine to aspartic acid at position 147 in exon 6 (p.147G>D) (FIG. 1). Interestingly, the G147D genetic variant of inhibitor-1 was identified only in black subjects with an allele frequency of 5-6% in either the population with heart failure (n=288) or the normal (n=40) population (all heterozygotes with one homozygote). This variant was not observed in 671 white patients although 1 white normal subject of the 312 who were screened exhibited the G147D polymorphism (see Table 2).

TABLE 2
Racial Distribution of Inhibitor-1 G147D Polymorphism

| | Patients with HF | Normal controls | | |
|---|---|---|---|---|
| | n (%) | n (%) | OR (95% CI) | P Value |
| Blacks | | | | |
| GG | 259 (89.9) | 35 (87.5) | 1 | |
| GD + DD | 28 + 1 (10.1) | 5 + 0 (12.5) | 0.784 (0.285-2.158) | 0.636 |
| Total | 288 (100) | 40 (100) | | |
| Freq. of Allele D (%) | 5.21 | 6.25 | 0.824 (0.310-2.189) | 0.603 |
| Whites | | | | |
| GG | 671 (100) | 311 (99.7) | 1 | |
| GD + DD | 0 | 1 + 0 (0.3) | 0.155 (0.006-3.806) | 0.142 |
| Total | 671 (100) | 312 (100) | | |
| Freq. of Allele D (%) | 0 | 0.02 | 0.155 (0.006-3.806) | 0.404 |
| Others | | | | |
| GG | 4 | 9 | | |
| GD + DD | 0 | 0 | | |
| Total | 4 | 9 | | |
| Freq. of Allele D (%) | 0 | 0 | | |

In one embodiment of the present invention, a method is provided for diagnosing the presence of a polymorphism of human phosphatase-1 inhibitor-1 protein which causes a predisposition to impaired functioning of the β-adrenergic signaling pathway, wherein the method comprises determining the presence of the polymorphism in a biological sample from a human subject by means which detect the presence of the polymorphism, wherein the polymorphism comprises aspartic acid at position 147 of the phosphatase-1 inhibitor-1 protein and wherein the presence of the polymorphism is indicative of the predisposition. In a specific embodiment, the polymorphism results in phosphatase-1 inhibitor-1 encoding the polypeptide of SEQ ID NO:14.

The polymorphism can be detected in the biological sample by analyzing any genetic material, including DNA, RNA, and protein, from which one skilled in the art can identify the presence of the G147D polymorphism. One skilled in the art will understand that there are a variety of techniques available for detecting the presence of the polymorphism in the biological sample. See, for example, Current Protocols in Molecular Biology, 2007, John Wiley and Sons, Inc. (Red Book). In the case of DNA, the methods include, but are not limited to, amplifying the DNA fragment of interest using polymerase chain reactions and sequencing the fragment to assess the presence or absence of the polymorphism. In one aspect of the invention, RNA can be extracted from the biological sample, translated into DNA using known methods such as reverse transcriptase, providing DNA suitable for sequencing. Suitable means for detecting the presence of the polymorphism include, but are not limited to, allele-specific antibody-based assays, ELISA, PCR based methods, nucleic acid hybridization, nucleic acid sequencing, DNA microarrays, DNA bead arrays, mass spectrometry, primer extension, branched DNA, and enzymatic cleavage methods and the like.

Exemplary detection methods for determining the presence or absence of the G147D polymorphism include the following:

### Direct Sequencing-Assays

In one embodiment of the present invention, the polymorphism is detected using a direct sequencing technique. In these assays, DNA samples are first isolated from a subject using any suitable method. In some embodiments, the region of interest is cloned into a suitable vector and amplified by growth in a host cell (e.g., a bacteria). In other embodiments, DNA in the region of interest is amplified using PCR.

Following amplification, DNA in the region of interest (e.g., the region containing the polymorphism) is sequenced using any suitable method, including but not limited to manual sequencing using radioactive marker nucleotides, and automated sequencing. The results of the sequencing are displayed using any suitable method. The sequence is examined and the presence or absence of a given polymorphism is determined.

### PCR Assay

In some embodiments of the present invention, variant sequences are detected using a PCR-based assay. In some embodiments, the PCR assay comprises the use of oligonucleotide primers that hybridize only to the variant or wild type allele (e.g., to the region of polymorphism or mutation). Both sets of primers are used to amplify a sample of DNA. If only the mutant primers result in a PCR product, then the patient has the mutant allele. If only the wild-type primers result in a PCR product, then the patient has the wild type allele.

### Hybridization Assays

In one embodiment, variant sequences are detected using a hybridization assay. In a hybridization assay, the presence or absence of the polymorphism is determined based on the ability of the DNA from the sample to hybridize to a complementary DNA molecule (e.g., an oligonucleotide probe). A variety of hybridization assays using a variety of technologies for hybridization and detection are known in the art and available to the skilled artisan. These assays include, but are not limited to, direct detection of hybridization, detection of hybridization using DNA chip assays, (for example, GeneChip (Affymetrix, Santa Clara, Calif.)), and enzymatic detection of hybridization.

Other detection assays that are suitable for use in the present invention include, but are not limited to, enzyme mismatch cleavage methods (e.g., Variagenics, U.S. Pat. Nos. 6,110,684, 5,958,692, 5,851,770); polymerase chain reaction; branched hybridization methods (e.g., Chiron, U.S. Pat. Nos. 5,849,481, 5,710,264, 5,124,246, and 5,624,802); rolling circle replication (e.g., U.S. Pat. Nos. 6,210,884 and 6,183,960); NASBA (e.g., U.S. Pat. No. 5,409,818); molecular beacon technology (e.g., U.S. Pat. No. 6,150,097); E-sensor technology (Motorola, U.S. Pat. Nos. 6,248,229, 6,221,583, 6,013,170, and 6,063,573); INVADER assay, Third Wave Technologies; See e.g., U.S. Pat. Nos. 5,846,717, 6,090,543; 6,001,567; 5,985,557; and 5,994,069); cycling probe technology (e.g., U.S. Pat. Nos. 5,403,711, 5,011,769, and 5,660,988); Dade Behring signal amplification methods (e.g., U.S. Pat. Nos. 6,121,001, 6,110,677, 5,914,230, 5,882,867, and 5,792,614); ligase chain reaction (Barnay Proc. Natl. Acad. Sci USA 88, 189-93 (1991)); and sandwich hybridization methods (e.g., U.S. Pat. No. 5,288,609).

### Mass Spectroscopy Assays

In another embodiment, a MassARRAY system (Sequenom, San Diego, Calif.) is used to detect variant sequences (See e.g., U.S. Pat. Nos. 6,043,031; 5,777,324; and 5,605,798). DNA is isolated from blood samples using standard procedures. Next, specific DNA regions containing the polymorphism of interest, about 200 base pairs in length, are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization-Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than one thousandth of a second, enabling samples to be analyzed in a total of 3-5 second including repetitive data collection. The SpectroTYPER software then calculates, records, compares and reports the genotypes at the rate of three seconds per sample.

In a specific embodiment of the present invention, the detecting means comprises analyzing DNA, RNA, or protein from the biological sample. In a more specific embodiment of the invention, the detecting means comprises carrying out a polymerase chain reaction in a mixture containing the biological sample and a pair of primers, wherein the primers are selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12 or complements thereof, under conditions suitable for amplifying a polynucleotide. In another embodiment, the detecting means further comprises sequencing the polynucleotide to detect the presence or absence of the polymorphism.

In another embodiment of the invention, an isolated polypeptide of SEQ ID NO:14 is provided. In another embodiment, an isolated DNA comprising a nucleic acid encoding the polypeptide of SEQ ID NO:14 is provided.

In yet another embodiment, a cell transfected with the DNA comprising a nucleic acid encoding the polypeptide of SEQ ID NO:14 is provided, wherein the cell is an isolated cell or a cell in culture.

In another embodiment, a vector is provided comprising the DNA comprising a nucleic acid encoding the polypeptide of SEQ ID NO:14. In another embodiment, a cell transfected with the vector is provided, wherein the cell is an isolated cell or a cell in culture.

In still another embodiment of the invention, a method is provided for detecting a polymorphism in human protein phosphatase-1 inhibitor-1, wherein the polymorphism is associated with impaired functioning of the β-adrenergic signaling pathway in a human subject, wherein the method comprises analyzing a protein phosphatase-1 inhibitor-1 gene or gene expression product from a biological sample of the human subject. In one embodiment, the sequence from the protein phosphatase-1 inhibitor-1 gene in the biological sample is compared with the sequence of one or more wild-type protein phosphatase-1 inhibitor-1 gene sequences. In another embodiment, the polymorphism results in phosphatase-1 inhibitor-1 encoding the polypeptide of SEQ ID NO:14. In another embodiment, a kit is provided for carrying out the method, the kit comprising (1) reagents for performing a detection assay, wherein the detection assay is configured to specifically identify the presence or absence of the polymorphism in human protein phosphatase-1 inhibitor-1; and (2) instructions for performing the detection assay.

In another embodiment, a method for determining whether a human subject is a candidate for a therapy that mitigates or restores an impairment of β-adrenergic responsiveness caused by a defective phosphatase-1 inhibitor-1 gene is provided, wherein the method comprises the steps of:

(1) obtaining a biological sample from the human subject, wherein the biological sample comprises nucleic acid molecules comprising a phosphatase-1 inhibitor-1 gene; and

(2) detecting the presence or absence of a genetic polymorphism of the phosphatase-1 inhibitor-1 gene of said human subject wherein the polymorphism comprises aspartic acid at position 147 of the phosphatase-1 inhibitor-1 protein,

wherein the presence of the polymorphism identifies a human subject who is a candidate for a therapy that mitigates or restores an impairment of the β-adrenergic responsiveness caused by a defective phosphatase-1 inhibitor-1 gene.

In one embodiment, the therapy can be gene therapy. Gene therapy can be used in any suitable manner, such as to treat the defective phosphatase-1 inhibitor-1 gene itself or to treat another target, the treatment of which mitigates or restores an impairment of the β-adrenergic responsiveness caused by the defective phosphatase-1 inhibitor-1 gene. Such other targets can include, for example, but are not limited to, SERCA2a, protein phosphatase-I, and phospholamban. Gene therapy can be administered using any suitable approaches or methods which are known in the art, such as by administering a target gene of interest (e.g., a wild-type phosphatase-1 inhibitor-1 gene) into heart cells, e.g., cardiomyocytes, to effectuate the replacement of the defective gene with a functioning gene, thereby mitigating or restoring impaired in β-adrenergic responsiveness induced or caused by the defective phosphatase-1 inhibitor-1 gene in the treated cells.

Heart cells suitable for gene therapy, e.g., gene replacement therapy of the defective phosphatase-1 inhibitor-1 gene, include both in vitro and in vivo cells. For example, the heart cells can be in a heart of a subject. The method can be used to treat a subject having a cardiac disorder, e.g., heart failure. Where type 1 phosphatases are targeted, they can include, but are not limited to, PPlcα, PPlcβ, PPl cδ and PPlcγ.

In another embodiment, a suitable agent for gene therapy can include a nucleic acid that comprises a sequence encoding a protein that inhibits phosphatase activity, e.g., type 1 phosphatase activity. The agent can be administered in an amount effective to decrease phosphatase activity and/or increase β-adrenergic responsiveness in the treated cells.

Delivery of the agent can occur through a variety of suitable means. For example, the agent can be delivered using a viral particle, such as a virus or a virus-like particle. Suitable viral particles can be derived, for example, from an adeno-associated virus, an adenovirus, or a lentivirus. In one embodiment, the viral particle is introduced by an injection, such as a direct injection into the heart. More specifically, the injection can be a direct injection into the left ventricle surface. In another embodiment, the viral particle is introduced into a lumen of the circulatory system, such as a chamber or the lumen of the heart or a blood vessel of the heart of a subject. The agent can also be delivered using means other than a viral particle, such as a liposome or other non-viral delivery vehicle, including stem cell delivery.

When administration of gene replacement therapy occurs via viral particles that enter cardiac cells, the particle includes a nucleic acid encoding a non-viral protein, for example, a protein that decreases phosphatase activity or a protein that modulates cardiac activity via the β-adrenergic signaling pathway. The preparation can be a cell-free preparation, such as a pharmaceutical preparation suitable for introduction into a subject. In one embodiment, the preparation can also contain less than 10, 5, 1 , 0.1 , or 0.001% pfu of wild-type virus (i.e., virus that can replicate and that does not include a non-viral nucleic acid sequence). In another embodiment, the preparation is free of wild-type virus.

In other embodiments, the therapy to mitigate or restore an impairment of β-adrenergic responsiveness caused by a defective phosphatase-1 inhibitor-1 gene can include the administration of a therapeutically effective amount of an agent, such as a small molecule, an antibody, a siRNA, a miRNA, or an antisense RNA, the effect of which, directly or indirectly, mitigates or restores the impairment of β-adrenergic responsiveness caused by a defective phosphatase-1 inhibitor-1 gene.

For example, in one embodiment the administered agent can be an inhibitor of the activity or expression of protein phosphatase-1, thereby mitigating or restoring the impairment of the β-adrenergic responsiveness caused by the defective phosphatase-1 inhibitor-1 gene.

In another embodiment, the agent can be an inhibitor of the activity or expression of phospholamban, thereby mitigating or restoring the impairment of the β-adrenergic responsiveness caused by the defective phosphatase-1 inhibitor-1 gene. β-adrenergic responsiveness can be increased by down-regulating expression of phospholamban (PLN), which is the functional target of phosphatase inhibitors. Heart failure is in part due to dysfunction of the PLN-controlled sarcoplasmic reticulum Ca²⁺ ATPase pump (SERCA2a) resulting from PLN phosphorylation and/or reduced SERCA2a expression. Unphosphorylated PLN keeps the Ca²⁺ affinity of SERCA2a low, resulting in decreased SR Ca²⁺ uptake, slowed relaxation and decreased SR Ca²⁺ load.

Where RNAi is utilized as the therapeutic agent, it will be appreciated that RNAi is well-known in the art. RNA interference (RNAi) is a novel therapeutic which may be used to target PLN. RNAi is mediated by small interfering RNA (siRNA) molecules. These are pairs of short, double-stranded RNA molecules typically 19-29 base pairs in length, one strand of which is complementary to a section of mRNA. The targeted mRNA is degraded by the RISC complex. One approach to the generation of siRNA in vivo is the generation of so-called small hairpin RNA (shRNA), which is a natural precursor of siRNA. Such shRNA molecules can be generated by expression of a partially self-complementary RNA molecule from an expression cassette introduced into a eukaryotic cell.

Where microRNA is utilized as the therapeutic agent, it will be appreciated that miRNA is well-known in the art. miRNA are RNA molecules naturally synthesized from genomically encoded genes that, upon processing by cellular RNA-modifying activities, render short RNA hairpin structures with 11 base pair stem structures. miRNA also targets mRNA for degradation and is probably a mechanism of gene expression control within the cell.

In another embodiment, the therapy can include administering a therapeutically effective amount of an agent that increases or enhances the expression and/or activity of SERCA2a, e.g., by using an appropriate small molecule, an antibody, a siRNA, a miRNA, or an antisense RNA. Gene therapy can also be used to deliver a therapeutically effective amount of a SERCA2a gene to increase its level of expression, thereby mitigating or restoring the impairment of β-adrenergic responsiveness caused by a defective phosphatase-1 inhibitor-1 gene. Heart failure can be treated by targeting SERCA2a expression. Indeed, replacement and/or increase of in vivo levels of SERCA2a, by introducing a nucleic acid that comprises a sequence encoding SERCA2a, is a viable alternative for the treatment of heart failure.

In a specific embodiment, the gene therapy and/or other therapy is selected from the group consisting of phosphatase-1 inhibitor-1 gene replacement therapy, SERCA2 gene therapy and RNA interference (RNAi) or phospholamban inhibition therapy. In another embodiment, the method further comprises administering phosphatase-1 inhibitor-1 gene replacement therapy to the human subject.

In yet another embodiment, the therapeutic agent can be administered to the subject via a coated stent. A “stent” is a medical device configured for implantation in a body lumen to prevent or inhibit the closing of the lumen. For example, the agent can be within a viral particle and the viral particle is coated on one or more surfaces of the stent, specifically, a surface that contacts the blood vessel.

With respect to the manner of administering gene replacement therapy, the effective amount, manner, or mode can vary depending on the subject and may be tailored to the subject. It is within the purview of the skilled artisan to determine the appropriate delivery system and dose for a given subject.

### Combination Therapies

In certain embodiments of the invention, methods may involve co-administering with the gene therapy having a therapeutic effect on the proteins of the SR Ca-transport pathway a second therapeutic agent, such as a beta blocker, an ionotrope (e.g., beta agonists, such as, dobutmaine), a diuretic, ACE-I, All antagonist, BNP, Ca⁺⁺-blocker, alsosterone blockers, phosphodiesterase inhibitors (e.g., milrinone), vasodilators (e.g., Natrecor), angiotensin type 2, endothelin antagonists, calcium channel blocker, cytokine blockers/inhibitors, cardiotonics, anti-thrombotic, hormone antagonists, or an HDAC inhibitor. As used herein, such co-administered therapies for patients with heart failure may also be referred to as “traditional” or “standard” cardiac therapies. In addition, traditional therapies that can be co-administered include heart devices, including ICD and BiV pacers, depending on the level of heart failure. Such traditional therapies can also be administered alone to any subject diagnosed with having a polymorphism in a phosphatase-1 inhibitor-1 gene, such as the G147D polymorphism, without co-administration with another agent or gene therapy.

In one embodiment, the second therapeutic agent may be administered or taken at the same time as the gene therapy treatment, or either before or after gene therapy treatment. The second therapeutic agent may improve one or more symptoms of pathologic cardiac hypertrophy or heart failure such as providing increased exercise capacity, increased cardiac ejection volume, decreased left ventricular end diastolic pressure, decreased pulmonary capillary wedge pressure, increased cardiac output or cardiac index, lowered pulmonary artery pressures, decreased left ventricular end systolic and diastolic dimensions, decreased left and right ventricular wall stress, decreased wall tension and wall thickness, increased quality of life, and decreased disease-related morbidity and mortality.

### Kits

In another embodiment, the present invention provides kits for the detection of phosphatase-1 inhibitor-1 protein polymorphisms. The kits may contain reagents specific for the detection of mRNA or cDNA (e.g., oligonucleotide probes or primers). In specific embodiments, the kits contain other components to facilitate a detection assay, including but not limited to controls, directions for performing assays, and software for analysis and presentation of results. In some embodiments, individual probes and reagents for detection of phosphatase-1 inhibitor-1 protein polymorphisms are provided as analyte specific reagents. In other embodiments, the kits are provided as in vitro diagnostics.

In a specific embodiment, a kit is provided for detecting the presence or absence of a polymorphism of phosphatase-1 inhibitor-1, the kit comprising a pair of primers, each primer having at least 95% identity to a primer selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12 or complements thereof.

In a more specific embodiment, the kit comprises primers selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12 or complements thereof.

### EXAMPLES

The following examples are given by way of illustration, and are in no way intended to limit the scope of the present invention. Results are expressed as means±SD. Analysis of variance was used to compare data between WT and G147D groups in the isolated myocyte studies. In all analyses, P<0.05 was considered statistically significant.

### Example 1

### Adult Rat Ventricular Myocyte Isolation and Culture

Adult rat ventricular myocytes were isolated and cultured. Hearts from adult male Sprague-Dawley rats (8 wk old) were perfused with modified Krebs-Henseleit buffer (KHB) (118 mM NaCl, 4.8 mM KCl, 25 mM HEPES, 1.25 mM K2HPO4, 1.25 mM MgSO4, 11 mM glucose, 5 mM taurine, and 10 mM butanedione monoxime; pH 7.4) for 5 min. Subsequently, hearts were perfused with an enzyme solution [KHB containing 0.7 mg/ml collagenase type II (263 U/mg), 0.2 mg/ml hyaluronidase, 0.1% BSA, and 25 μM Ca]. Ten and 15 min later, 25 and 50 μM Ca, respectively, were added to the perfusion buffer, so that the Ca concentration was raised to 100 μM, and perfusion was continued for another 5 min. Finally, left ventricular tissue was excised, minced, pipette dissociated, and filtered through a 24082 m screen. Cells were harvested and resuspended in KM including 1 mM Ca and 1% BSA. After a brief centrifugation, the cells were resuspended in KHB including 1.8 mM Ca and 1% BSA, centrifuged briefly again, and resuspended in Dulbecco's modified Eagle's medium containing 2 mg/ml BSA, 2 mM L-camitine, 5 mM creatine, 5 mM taurine, 1.8 mM Ca, 100 IU/ml penicillin, and 100 μg/ml streptomycin. Cells were then counted and plated on laminin-coated glass coverslips or dishes.

### Example 2

### Adenovirus-Mediated Gene Transfer

Recombinant adenovirus carrying empty vector, wild-type (WT) G147 inhibitor-1, or D147 inhibitor-1 was generated by using the Adeno-X expression system kit (Clontech, Mountain View, Calif.). All of the adenoviruses also expressed GFP as an indicator of transfection efficiency. Two hours after isolation and culture, myocytes seeded on coverslips or dishes were infected with adenovirus in diluted media, at a multiplicity of infection of 500, for 2 h before the addition of a suitable volume of culture media. Twenty-four hours later, the myocytes were washed in KHB including 1 mM Ca and harvested for quantitative immunoblotting or use in the experiments outlined below.

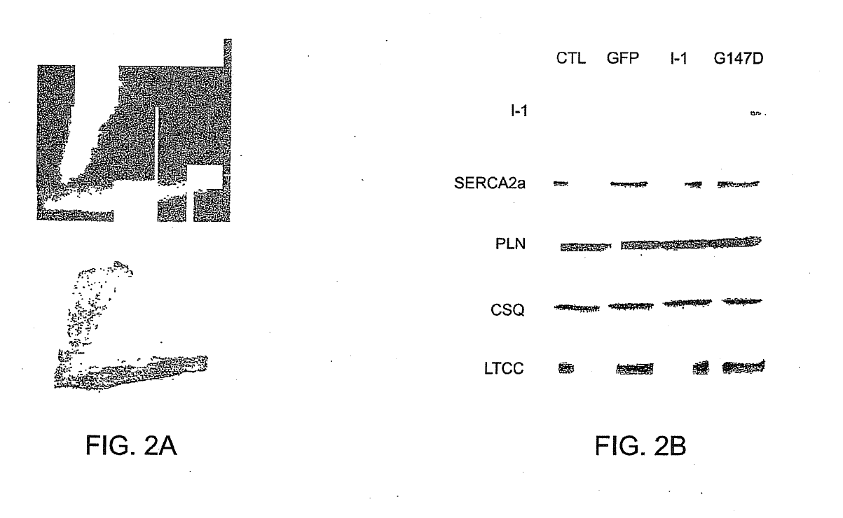

GFP fluorescence indicated >90% infectivity (FIG. 2A). Quantitative immunoblotting revealed that cardiomyocytes expressed comparable levels of WT inhibitor-1 and G147D inhibitor-1 (FIG. 2B, C) at 24 h post-transfection. Importantly, overexpression of inhibitor-1 or its mutant did not alter the levels of the major regulatory proteins related to cardiac calcium homeostasis (FIG. 2B), indicating minimal compensatory changes in the adult cardiomyocytes.

### Example 3

### Contractile Parameter Measurements

Myocytes that adhered to the coverslips were bathed in temperature (37° C.)—equilibrated KHB containing 1 mM Ca for 20 min. The myocyte suspension was then placed in a Plexiglas chamber, which was positioned on the stage of an inverted epifluorescence microscope (Diaphot 200; Nikon, Tokyo, Japan). Myocyte contraction was field-stimulated by a Grass S5 stimulator (0.5 Hz, square. waves; Grass Technologies, An Astro-Med, Inc., West Warwick, R.I., USA), and contractions were videotaped and digitized on a computer. A video edge motion detector (Crescent Electronics, Windsor, ON, Canada) was used to measure myocyte length and cell shortening, from which the percent fractional shortening (% FS) and maximal rates of contraction and relaxation (±dL/dt) were calculated. To investigate the response of adult rat cardiomyocytes to isoproterenol, a maximal concentration of isoproterenol (100 nM) was added to the KHB (including 1 mM Ca) in the Plexiglas chamber, and the above-mentioned measurements were repeated. All data were analyzed using software from Felix 1.1 software (Photon Technology International, Birmingham, N.J., USA) and IonWizard (IonOptix Corp., Milton, Mass., USA).

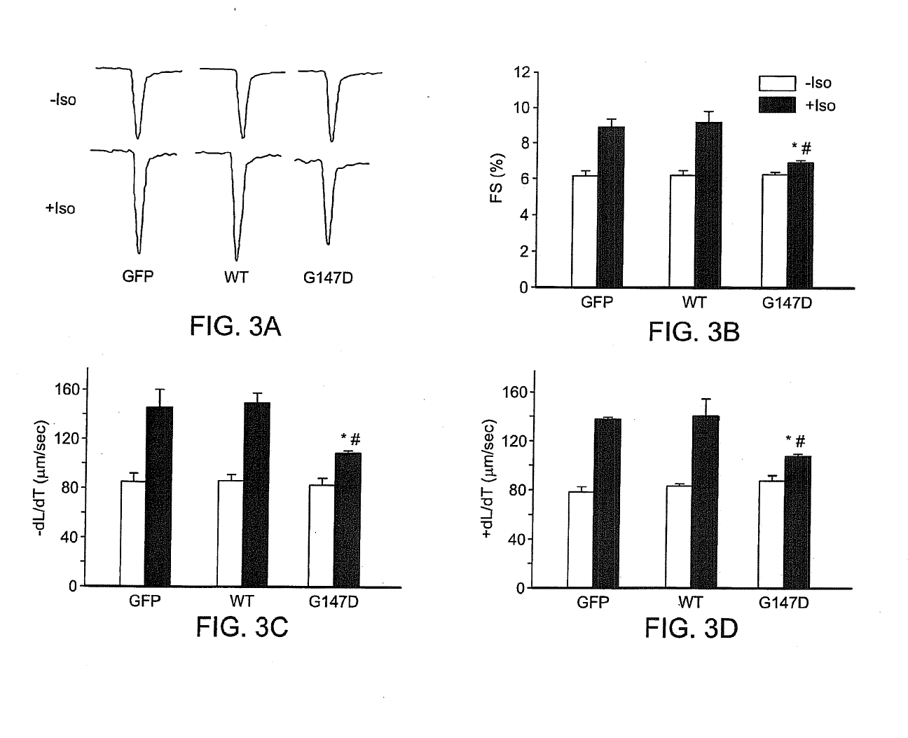

Mechanical properties of the infected cardiomyocytes indicated that overexpression of WT inhibitor-1 or the G147D mutant did not alter cellular contractility, as revealed by fractional shortening, contracting velocity, and relaxing velocity under basal conditions (FIG. 3).

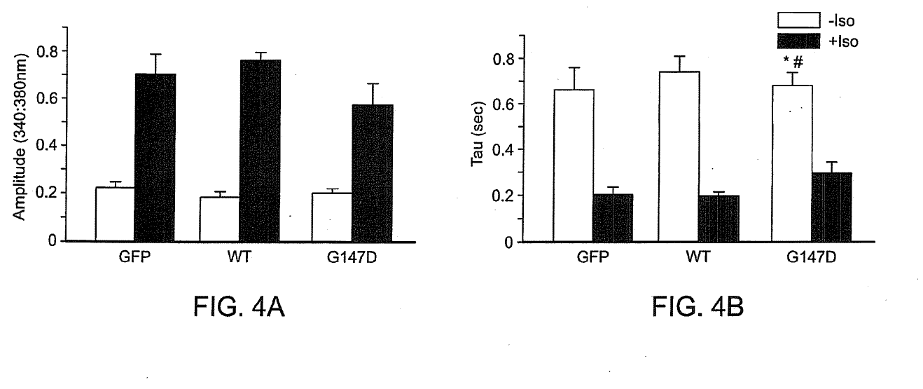

On stimulation with 100 nM isoproterenol, cardiomyocytes infected with control or WT inhibitor-1 viruses displayed enhanced contractility, demonstrated by increased fractional shortening (FIG. 3B), accelerated contraction (FIG. 3C), and enhanced relaxation (FIG. 3D). However, the effect of isoproterenol was significantly blunted in cardiomyocytes with overexpression of D147 inhibitor-1 (FIG. 3). Although the contractile parameters in D147 cells were significantly increased by isoproterenol treatment, these values remained depressed by 24-28% compared with WT inhibitor-1-stimulated cells. Similar findings were observed in calcium kinetic parameters of the G147D inhibitor-1-stimulated cardiomyocytes (FIG. 4). Furthermore, a lower isoproterenol dose (10 nM) revealed differences similar to those observed in 100 nM as described above. Thus, the G147 inhibitor-1 variant attenuated the stimulatory responses of isoproterenol on calcium kinetics and mechanics of adult cardiomyocytes.

### Example 4

### Regulation of Protein Phosphorylation

Alterations in the phosphorylation and/or total level of proteins were analyzed from whole heart homogenates, with the exception of MyBP-C, which was analyzed from a myofibril protein preparation, using Western blotting. 2-70 μg of protein were separated on polyacrylamide gels (6-15%) and transferred to nitrocellulose membranes. The membranes were incubated with primary antibodies, which were visualized by peroxidase-conjugated secondary antibodies (Amersham Biosciences) and enhanced chemiluminescence (Supersignal West Pico Chemiluminescent, Pierce). The bands were then quantified with densitometry, using ImageQuant 5.2 (GE Healthcare, Little Chalfont, UK). The antibodies were obtained from the sources listed: pSer16-PLN, pThr17-PLN and pSer2808-RyR (Badrilla), PLN (Upstate), RyR and actin (Sigma), pSer22/Ser23-TnI and TnI (Research Diagnostics Inc.), pSer282-MyBP-C (custom-made commercially, ProSci Inc.), MyBP-C (home-made, references 2, 3), I-1 and SERCA2a (custom-made commercially, Affinity Bioreagents), CSQ (Affinity Bioreagents), PDI (Alexis Biochemicals), Grp78 (Santa Cruz Biotechnology) and Ire1α (Abcam).

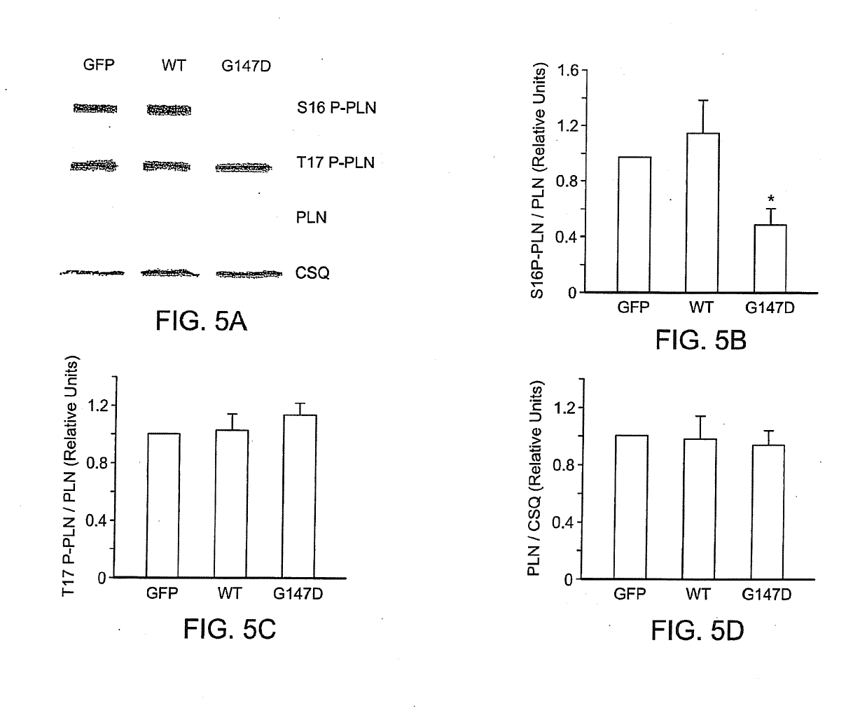

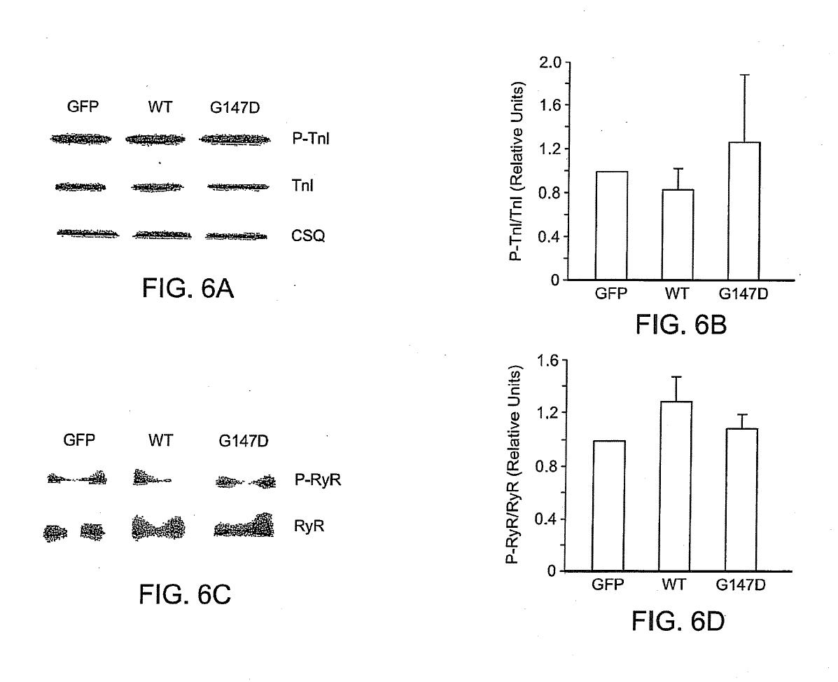

Under basal conditions, phosphorylation of PLN was not detectable in isoproterenol-treated adult rat cardiomyocytes. In the presence of isoproterenol, phosphorylation of PLN was similarly increased in both WT inhibitor-1-expressing and control cells, in agreement with the data for contractility. However, the level of phosphorylated PLN on serine 16 was depressed by ˜50% in the G147 inhibitor-1-infected myocytes (FIG. 5). Interestingly, the levels of PLN phosphorylation on threonine 17 were similar among the three groups. In addition, phosphorylation levels of troponin I (serine 22/23) and RyR (serine 2809) were also examined. As expected, the phosphorylation levels of these two proteins were increased by isoproterenol treatment. However, there were no significant differences observed between the three groups in the presence of isoproterenol (FIG. 6). Thus, the attenuated stimulatory responses to isoproterenol were associated mainly with decreased PKA phosphorylation of PLN in the G147D inhibitor-1-infected cardiomyocytes.

All documents cited are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for diagnosing the presence of a polymorphism of human phosphatase-1 inhibitor-1 protein which causes a predisposition to impaired functioning of the β-adrenergic signaling pathway, wherein the method comprises determining the presence of the polymorphism in a biological sample from a human subject by means which detect the presence of the polymorphism, wherein the polymorphism comprises aspartic acid at position 147 of the phosphatase-1 inhibitor-1 protein and wherein the presence of the polymorphism is indicative of said predisposition.

2. The method of claim 1 wherein the polymorphism results in a phosphatase-1 inhibitor-1 having the amino acid sequence of SEQ ID NO:14.

3. The method of claim 2 wherein the detecting means comprises analyzing DNA, RNA, or protein from the biological sample.

4. The method of claim 2 wherein the detecting means comprises carrying out a polymerase chain reaction in a mixture containing the biological sample and a pair of primers, wherein the primers are selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12 or complements thereof, under conditions suitable for amplifying a polynucleotide.

5. The method of claim 4 wherein the detecting means further comprises sequencing the polynucleotide to detect the presence or absence of the polymorphism.

6. An isolated phosphatase-1 inhibitor-1 protein having the amino acid sequence of SEQ ID NO:14.

7. An isolated DNA comprising a nucleic acid sequence encoding a phosphatase-1 inhibitor-1 having the amino acid sequence of SEQ ID NO:14.

8. A cell transfected with the DNA of claim 7, wherein the cell is an isolated cell or a cell in culture.

9. A vector comprising the isolated DNA of claim 7.

10. A cell transfected with the vector of claim 9, wherein the cell is an isolated cell or a cell in culture.

11. A method for detecting a polymorphism in human protein phosphatase-1 inhibitor-1, wherein the polymorphism is associated with impaired functioning of the β-adrenergic signaling pathway in a human subject, wherein the method comprises analyzing a protein phosphatase-1 inhibitor-1 gene or gene expression product from a biological sample of the human subject.

12. The method of claim 11 wherein the sequence from the protein phosphatase-1 inhibitor-1 gene in the biological sample is compared with the sequence of one or more wild-type protein phosphatase-1 inhibitor-1 gene sequences.

13. The method of claim 12 wherein the polymorphism results in phosphatase-1 inhibitor-1 having the amino acid sequence of SEQ ID NO:14.

14. A method for determining whether a human subject is a candidate for a therapy that mitigates an impairment of β-adrenergic responsiveness caused by a defective phosphatase-1 inhibitor-1 gene, the method comprising the steps of:
(1) obtaining a biological sample from the human subject, wherein the biological sample comprises a nucleic acid molecule encoding a phosphatase-1 inhibitor-1 gene; and
(2) detecting the presence or absence of a genetic polymorphism of the phosphatase-1 inhibitor-1 gene of said human subject,
wherein the presence of the polymorphism identifies a human subject who is a candidate for a therapy that mitigates an impairment of the β-adrenergic responsiveness caused by a defective phosphatase-1 inhibitor-1 gene.

15. The method of claim 14, wherein the genetic polymorphism of the phosphatase-1 inhibitor-1 gene comprises aspartic acid (D) in place of glycine (G) at position 147 of the phosphatase-1 inhibitor-1 protein.

16. The method of claim 14, wherein the therapy is to administer gene therapy such that the impairment of the β-adrenergic responsiveness is mitigated.

17. The method of claim 16, wherein the gene therapy comprises gene replacement therapy of the defective phosphatase-1 inhibitor-1 gene with a non-defective phosphatase-1 inhibitor-1 gene, thereby mitigating the impairment of the β-adrenergic responsiveness caused by the defective phosphatase-1 inhibitor-1 gene.

18. The method of claim 16, wherein the gene therapy comprises administering a therapeutically effective amount of a vector encoding SERCA2a, thereby mitigating the impairment of the β-adrenergic responsiveness caused by the defective phosphatase-1 inhibitor-1 gene.

19. The method of claim 14, wherein the therapy is to administer a therapeutically effective amount of an agent to mitigate the impairment of the β-adrenergic responsiveness.

20. The method of claim 19, wherein the agent is an inhibitor of the activity or expression of protein phosphatase-1, thereby mitigating the impairment of the β-adrenergic responsiveness caused by the defective phosphatase-1 inhibitor-1 gene.

21. The method of claim 19, wherein the agent is an inhibitor of the activity or expression of phospholamban, thereby mitigating the impairment of the β-adrenergic responsiveness caused by the defective phosphatase-1 inhibitor-1 gene.

22. The method of claim 19, wherein the inhibitor agent is a small molecule, an antibody, a siRNA, a miRNA, or an antisense RNA.

23. The method of claim 16, wherein, in addition to the gene therapy, the therapy comprises co-administering a traditional treatment for heart failure.

24. The method of claim 19, wherein, in addition to administering the agent, the therapy comprises co-administering a traditional treatment for heart failure.

25. The method of claim 14, wherein the therapy comprises co-administering a traditional treatment for heart failure.

26. The method of claim 23, 24 or 25, wherein the traditional treatment for heart failure is selected from the group consisting of co-administering a therapeutically effective amount of an ACE inhibitor, a beta blocker, a diuretic, an alsosterone blocker, an ionotrope, a phosphodiesterase inhibitor, a calcium blocker, an HDAC inhibitor, and a All antagonist.

27. The method of claim 23, 24 or 25, wherein the traditional treatment for heart failure is treatment with an ICD device or BiV pacer.

28. A kit for carrying out the method of claim 11 or 14, the kit comprising:
(1) a reagent for performing a detection assay, wherein the detection assay is configured to specifically identify the presence or absence of a polymorphism in human protein phosphatase-1 inhibitor-1; and
(2) instructions for performing the detection assay.

29. The kit of claim 28, wherein the human protein phosphatase-1 inhibitor-1 has the amino acid sequence of SEQ ID NO:14.

30. A kit for detecting the presence or absence of a polymorphism of phosphatase-1 inhibitor-1, the kit comprising a pair of primers, each primer having at least 95% identity to a primer selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12 or complements thereof.

31. The kit of claim 29, wherein the primers are selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12 or complements thereof.


SEQUENCE LISTING (16 sequences)

SEQ ID NO: 1 - DNA, 26 bp, Artificial Sequence
  Description of Artificial Sequence Synthetic primer
caaaactccgaggacactgaggtatc

SEQ ID NO: 2 - DNA, 24 bp, Artificial Sequence
  Description of Artificial Sequence Synthetic primer
caaaagggcgcactgctaagggag

SEQ ID NO: 3 - DNA, 21 bp, Artificial Sequence
  Description of Artificial Sequence Synthetic primer
ggcctccagctgcattaacat

SEQ ID NO: 4 - DNA, 21 bp, Artificial Sequence
  Description of Artificial Sequence Synthetic primer
gttcttcttccatctagcgcc

SEQ ID NO: 5 - DNA, 23 bp, Artificial Sequence
  Description of Artificial Sequence Synthetic primer
caaggacgggactagatgcagag

SEQ ID NO: 6 - DNA, 21 bp, Artificial Sequence
  Description of Artificial Sequence Synthetic primer
gttcactcatgcacatttggg

SEQ ID NO: 7 - DNA, 21 bp, Artificial Sequence
  Description of Artificial Sequence Synthetic primer
ttgctcaccattgtagtctcc

SEQ ID NO: 8 - DNA, 21 bp, Artificial Sequence
  Description of Artificial Sequence Synthetic primer
cccagctagtcagtggcaaaa

SEQ ID NO: 9 - DNA, 22 bp, Artificial Sequence
  Description of Artificial Sequence Synthetic primer
cctccttgttcagatctcagtg

SEQ ID NO: 10 - DNA, 19 bp, Artificial Sequence
  Description of Artificial Sequence Synthetic primer
ccttgagacagtttttgcc

SEQ ID NO: 11 - DNA, 22 bp, Artificial Sequence
  Description of Artificial Sequence Synthetic primer
ccctgttggtcttgcctgattg

SEQ ID NO: 12 - DNA, 22 bp, Artificial Sequence
  Description of Artificial Sequence Synthetic primer
cctactgctctcacccattcca

SEQ ID NO: 13 - protein, 171 aa, Homo sapiens
MEQDNSPRKIQFTVPLLEPHLDPEAAEQIRRRRPTPATLVLTSDQSSPEIDEDRIPNPHLKSTLAMSPRQRKKMTRITPTMKELQMMVEHHLGQQQQGEEPEGAAESTGTQESRPPGIPDTEVESRLGTSGTAKKTAECIPKTHERGSKEPSTKEPSTHIPPLDSKGANSV

SEQ ID NO: 14 - protein, 171 aa, Homo sapiens
MEQDNSPRKIQFTVPLLEPHLDPEAAEQIRRRRPTPATLVLTSDQSSPEIDEDRIPNPHLKSTLAMSPRQRKKMTRITPTMKELQMMVEHHLGQQQQGEEPEGAAESTGTQESRPPGIPDTEVESRLGTSGTAKKTAECIPKTHERDSKEPSTKEPSTHIPPLDSKGANSV

SEQ ID NO: 15 - DNA, 516 bp, Homo sapiens
atggagcaagacaacagcccccgaaagatccagttcacggtcccgctgctggagccgcaccttgaccccgaggcggcggagcagattcggaggcgccgccccacccctgccaccctcgtgctgaccagtgaccagtcatccccagagatagatgaagaccggatccccaacccacatctcaagtccactttggcaatgtctccacggcaacggaagaagatgacaaggatcacacccacaatgaaagagctccagatgatggttgaacatcacctggggcaacagcagcaaggagaggaacctgagggggccgctgagagcacaggaacccaggagtcccgcccacctgggatcccagacacagaagtggagtcaaggctgggcacctctgggacagcaaaaaaaactgcagaatgcatccctaaaactcacgagagaggcagtaaggaacccagcacaaaagaaccctcaacccatataccaccactggattccaagggagccaactcggtctga

SEQ ID NO: 16 - DNA, 516 bp, Homo sapiens
atggagcaagacaacagcccccgaaagatccagttcacggtcccgctgctggagccgcaccttgaccccgaggcggcggagcagattcggaggcgccgccccacccctgccaccctcgtgctgaccagtgaccagtcatccccagagatagatgaagaccggatccccaacccacatctcaagtccactttggcaatgtctccacggcaacggaagaagatgacaaggatcacacccacaatgaaagagctccagatgatggttgaacatcacctggggcaacagcagcaaggagaggaacctgagggggccgctgagagcacaggaacccaggagtcccgcccacctgggatcccagacacagaagtggagtcaaggctgggcacctctgggacagcaaaaaaaactgcagaatgcatccctaaaactcacgagagagacagtaaggaacccagcacaaaagaaccctcaacccatataccaccactggattccaagggagccaactcggtctga